(19) United States
(12) Patent Application Publication
(10) Pub. No.: US 2026/0369419 A1
(43) Pub. Date: Nov. 19, 2026

(54) AGENTIC APPROACH TO USER RESPONSE CLASSIFICATION

(71) Applicant: Thrive AI Health, Inc., Oakland, CA (US)

(72) Inventors: DeCarlos Love, Alameda, CA (US); Melissa Christina Bennett-Pinnock, Lake Balboa, CA (US); Paras Unadkat, San Francisco, CA (US); Marc Ammann, Portland, OR (US)

(21) Appl. No.: 19/672,412

(22) Filed: May 8, 2026

Related U.S. Application Data
(60) Provisional application No. 63/805,847, filed on May 14, 2025.

(51) Int. Cl. G16H 50/30; G06Q 10/0631

(52) U.S. Cl. CPC G16H 50/30; G06Q 10/06316

(57) ABSTRACT

An LLM-based agent receives a natural-language query requesting wellness actions and generates a set of tasks, each associated with a set of context. The LLM agent selects a first generative AI tool to perform the first task. The first tool processes the first task using a first context including a transcript of a conversation with the user. The agent selects a second generative AI tool and sends the first output and a second set of context to second tool, the first output including user characteristics. The second tool processes the second task using the second context, including user's wellness data, and the first result. The agent selects a third generative AI tool and third context and sends a second result and third context to the third tool, the second result including the user's physical abilities and health metrics. The third tool generates recommendations for one or more wellness actions.

### CROSS REFERENCE TO RELATED APPLICATIONS

This application claims the benefit of U.S. Provisional Patent Application Ser. No. 63/805,847, filed May 14, 2025, which is incorporated by reference herein in its entirety.

### BACKGROUND

Generative Artificial Intelligence (AI) models are advanced machine learning systems that create new content, such as text, images, audio, or code, by learning patterns from vast amounts of data. These models, like ChatGPT for text generation, use deep learning techniques to generate human-like outputs based on prompts or input data. Many models with various capabilities are available to users today, but such models may be easily confused or unable to complete processing when asked to complete complex tasks. For example, in the context of health coaching, a model may be used to generate wellness actions for a user to do. However, to personalize the actions to the user, the model would need to consider many wellness dimensions, such as nutrition, exercise, psychology, etc., as well as health metrics, preferences, and even mood of the user. This is too much information for conventional models to process as conventional AI models often struggle with context overload, reasoning limitations, and poor long-term memory retention, which leads to generic, unreliable, or inconsistent outputs.

Health records are often an extensive, multimodal collection of information. The span of information that can be recorded and associated with a subject's wellness extends across a vast variety of wellness specialties, each with their own associated metrics and procedures to assess wellness. A physical trainer might track exercise data using a wearable device to assess training progress of the user, while a cardiologist might impose restrictions for various exercise plans based on medical charting for the user. Similarly, the user might track sleep quality themselves using a wearable device and a dietitian may track calorie intake logs of a user to which they assigned a dietary plan.

Even a single factor in a user's overall wellness (e.g., sleep quality or an underlying disease) can have an outsize effect their wellness that impacts every other factor. Wellness data can be compressed to generally reduce its size using various data aggregation techniques, therefore reducing the amount of context to be loaded into an agentic system. However, the original, uncompressed health records may contain relatively small details that have outsized influences on wellness outcomes for a subject. Compressing health records or performing filtering actions of the user wellness data repository can have severe consequences.

The amount of context that an LLM can process is determined by its context window. The LLM performs tasks (e.g., reasoning or making predictions) based on data loaded into the context window. As such, the LLM cannot consider the small details, which may be lost due to compression, that are not present in its context window. In practice, it is not feasible to load an extensive user history into the context window of a single LLM. Even if this was feasible, introducing data into the context window of an LLM that is irrelevant to a prompt can result in noisy outputs and hallucinations. Further, processing latency tends to increase as the context window increases. Therefore, there exists a technical problem of determining optimal context, from a huge corpus of candidate context that exceeds a context window, that is relevant to a user's wellness without exceeding the context window of any one model used to determine a personal wellness plan for a user.

### SUMMARY

The following agentic system improves the abilities of existing generative AI models by employing a multi-agent architecture of generative AI models, such as large language models (LLMs) to complete complex tasks, such as determining a personalized wellness program of wellness actions. For instance, the LLMs in an agentic system may communicate outputs to one another such that each may be used for a smaller, discrete task (e.g., a specific aspect of user care) that is necessary to complete the overall task of creating a personalized wellness program. Each LLM may be capable of determining a next LLM to send its output to, allowing the LLMs to operate autonomously and without user input beyond an initial natural language query input to the agentic system. By working together in a chain-like architecture, the LLMs may each focus on smaller tasks, thus reducing the likelihood of producing hallucinations or other confusing outputs, while still completing the complex task.

Each agent of the agentic system is equipped with tools that extend its capabilities beyond those of conventional agents. The tools enable each agent to perform real-time data retrieval, computation, health analytics, and decision-making, such that the agent may generate precise, fact-based recommendations. The agents may access a shared tool ecosystem at the agentic system that allows the agents to access memory and application programming interfaces (APIs), which facilitates seamless collaboration between the agents while maintaining efficiency of the agentic system and personalization for wellness programming.

The agentic system employs a single graph approach where agents are integrated into a unified execution framework, which is a simplified system compared to one that uses standalone agents and redundant routing logic. For instance, the router agent may determine whether to continue a conversation with a current agent, transition the conversation to another agent, or conclude the conversation. By using this approach, the agentic system determines and loads subsets of context for LLMs that include relevant wellness data for the various tasks of the wellness plan.. Context chaining between agents and selective allocation of subsets of context (e.g., using a routing agent) optimally populates the context window of each LLM down a task chain with wellness data that is relevant to the task and obscures irrelevant data that may cause hallucinations and latency. As such, LLMs perform tasks such as reasoning and retrieval in view of relevant wellness data and pass the results to downstream LLMs. The downstream LLMs can receive processed outputs without the need to retrieve related wellness data from the original source. Therefore, the agentic system solves the technical problem of maintaining an accurate context describing a user's wellness in the technical field of generating personalized wellness programming.

Put another way, the agentic system uses an agent to determine a set of modularized tasks that build towards completion of an overall task. The agentic system breaks down high-level wellness programming into smaller, manageable tasks, each handed by a dedicated agent of the agentic system. The agents of the agentic system communicate dynamically with one another by passing outputs to the next relevant agent based on context about a user and the user's specific wellness programming, ensuring a structured flow of decision-making between the agents. Thus, by modularizing every overall task into a set of tasks, the agentic system prevents the LLMs from being given too complex of a task or too much context and allows the overall system to perform complex reasoning and planning tasks. The LLMs may complete the set of tasks in a chain-like manner-e.g., a first LLM processes a first task, the output of which is sent to an LLM that processes a second task, the output of which is sent to a third LLM that processes a third task, and so forth. In some embodiments, the agentic system may use a router agent to decide which sets of LLMs should be invoked in concert to successfully perform a given task and for selective persistent storage and retrieval of context needed for tasks processed at each LLM. For example, a router LLM may track the progress of the set of tasks through the chain of LLMs and input context to complete a task at a respective LLM when the LLM receives an output of a previous task. In some embodiments, each LLM determines what context the next LLM needs to complete its task and sends the context with its output to the next LLM.

### BRIEF DESCRIPTION OF DRAWINGS

FIG. 1A is a block diagram of an overall system environment illustrating a data movement system, in accordance with one or more illustrative embodiments.

FIG. 1B is an example system architecture for an agentic system, in accordance with one or more illustrative embodiments.

FIG. 2 is a flowchart showing an exemplary process for deploying a personalized wellness agent, in accordance with one or more illustrative embodiments.

FIG. 3 is a high-level block diagram of a computer for implementing different entities illustrated in FIGS. 1A and 1B, in accordance with one or more illustrative embodiments.

### DETAILED DESCRIPTION

Figures (FIG.) and the following description relate to various embodiments by way of illustration only. It should be noted that from the following discussion, alternative embodiments of the structures and methods disclosed herein will be readily recognized as viable alternatives that may be employed without departing from the principles discussed herein. Reference will now be made in detail to several embodiments, examples of which are illustrated in the accompanying figures. It is noted that wherever practicable similar or like reference numbers may be used in the figures and may indicate similar or like functionality.

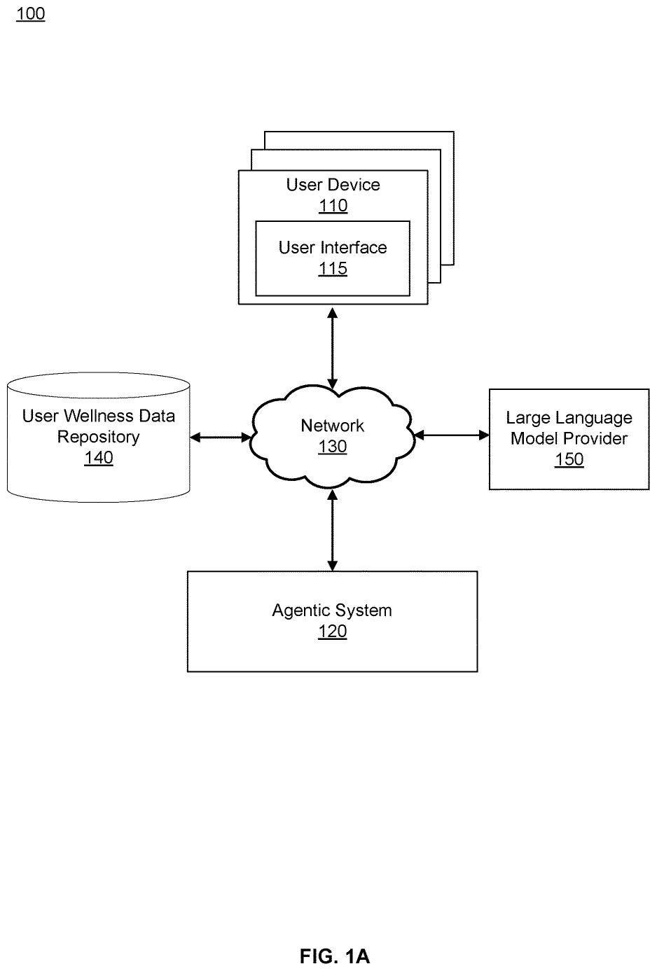

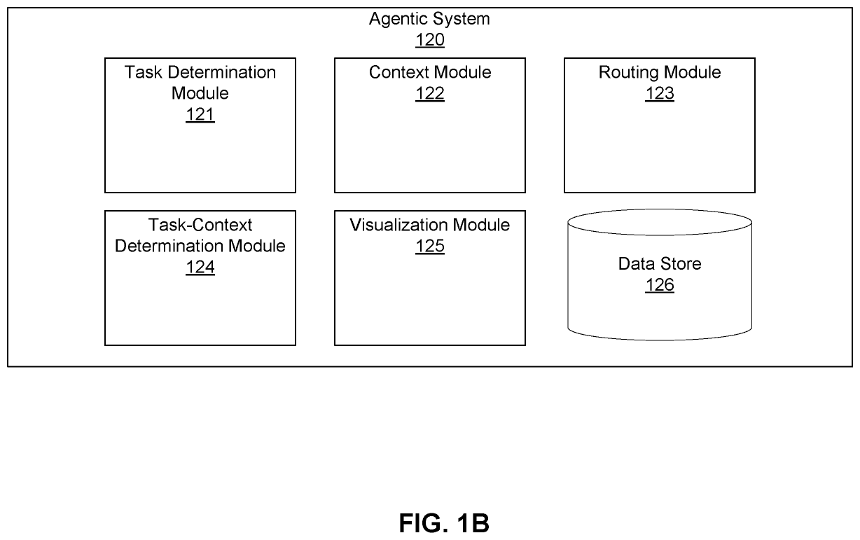

FIG. 1A is a block diagram of an overall system environment illustrating an agentic system 120, according to one or more illustrative embodiments. As shown in FIG. 1A, the overall system environment includes the agentic system 120, one or more user devices 110, a network 130, a user wellness data repository 140, and a large language model provider 150. Other embodiments may use more or fewer or different systems than those illustrated in FIG. 1A. Functions of various modules and systems described herein can be implemented by other modules and/or systems than those described herein.

A user device 110 (also referred to herein as a “client device”) is a computing system used by users to interact with the agentic system 120. A user interacts with the agentic system 120 using a user device 110 that executes client software, e.g., a web browser or a client application, to connect to the agentic system 120. The user device 110 displayed in these embodiments can include, for example, a mobile device (e.g., a laptop, a smart phone, or a tablet with operating systems such as Android or Apple IOS etc.), a desktop, a smart automobile or other vehicle, wearable devices, a smart TV, and other network-capable devices.

The user device 110 can present media content provided by the agentic system 120. In some embodiments, the user device 110 includes an embedded video player such as, for example, the FLASH player from Adobe Systems, Inc., or any other player adapted for various media formats used in the agentic system 120.

The network 130 facilitates communication between the user devices 110 and the agentic system 120. The network 130 is typically the Internet, but may be any network, including but not limited to a LAN, a MAN, a WAN, a mobile wired or wireless network, a cloud computing network, a private network, or a virtual private network.

The agentic system 120 may include one or more modules or components that are used to implement the methods and system described below. Other features of the agentic system 120, such as firewalls, load balancers, authentication servers, application servers, failover servers, and site management tools are not shown so as to more clearly illustrate the features of the agentic system 120. The illustrated components of the agentic system 120 can be implemented as single or multiple components of software or hardware. In general, functions described in one embodiment as being performed by components of the agentic system 120 can also be performed by one or more user devices 110 or other systems in other embodiments if appropriate.

The agentic system 120 may retrieve user wellness data and store the user wellness data at a local database, a database accessible via the network 130, or a user wellness data repository 140. The agentic system 120 may receive user wellness data from one or more user devices 110 connected via the network 130. For instance, the agentic system 120 may provide a mobile application or web browser configured to receive interactions indicative of user wellness data and cause a user device 110 to present a user interface 115 that enables a user to enter user wellness data or to configure the user device 110 to use one or more sensors to capture user wellness data. The agentic system may also receive user wellness data from other devices, such as a smart watch or glucose monitor, configured to capture user wellness data via one or more sensors or user device 110 of other users, such as medical professionals or coaches. In some embodiments, the agentic system 120 receives approval by a user retrieving and storing a user's user wellness data. For example, the user wellness data repository 140 may comprise sources of data such as a 3rd-party database associated with a smart device that the user uses to capture wellness data.

User wellness data is stored in the user wellness data repository 140 (or alternatively, the data store 126 of the agentic system 120). In some embodiments, the data store 126 comprises a portion or all of the contents of the user wellness data repository 140. User wellness data may be health metrics describing a user's daily activity (e.g., steps taken, calories burned, distance walked, etc.), a user's heart health (resting heart rate, cardiovascular fitness score, etc.), sleep (duration, stages, etc.), stress (electrodermal activity, stress score, etc.), medical data (electronic health reports include diagnoses, medications, lab results, etc.), fitness data (mile time, maximum number of push-ups, barbell weight for squats, etc.), and other health or wellness-related metrics (e.g., blood oxygen saturation, skin temperature, breathing rate, food/nutrient intake, etc.). User wellness data may also include other metrics captured from devices associated with a user, such as wearable technology, mobile phones, and the like.

The agentic system 120 may access one or more off-the-shelf (e.g., ChatGPT, LLaMA, etc.) or internally constructed LLMs in a system of LLMs from an LLM provider 150. The agentic system 120 may receive a natural language query that the agentic system 120 parses to determine one or more tasks described in the query. The natural language query may be textual or voice input from an external use or generated by an LLM.

FIG. 1B illustrates an example system architecture for an agentic system, in accordance with one or more illustrative embodiments. Agentic system 120 includes a task determination module 121, a context module 122, a routing module 123, a task-context determination module 124, a visualization module 125, and a data store 126. Alternative embodiments may include more, fewer, or different components from those illustrated in FIG. 1B and the components may be arranged or configured differently from the embodiment illustrated in FIG. 1B in any manner that achieves the functionality disclosed herein.

The agentic system 120 determines wellness actions for users to take. A wellness action describes an activity for a user to do to improve their user wellness data or a specific health metric from the user wellness data. As an example, a wellness action may be associated with a time period that ends at a goal date, and the agentic system 120 may present the wellness action to the user to suggest the user complete the wellness action during the time period until the goal date. The agentic system may send wellness actions for display at a user interface 115, which may also include interactive elements that allow a user to input whether she did all or part of the wellness action. In some embodiments, the agentic system automatically determines whether a user completed a wellness action based on an analysis of the user's user wellness data during the time period. The agentic system stores associated wellness actions in association with a user's identifier, the time period, the goal date, determinations of level of completion of the wellness action, and associated user wellness data.

The agentic system 120 determines wellness actions by leveraging a system of LLMs (and, in some embodiments, other generative AI models and systems). At a high level, the system of LLMs (henceforth referred to as the LLM system) may use a user's user wellness data as context about the user to determine baselines for the user and work together in a cascading system to determine wellness actions to recommend to the user. Each LLM (also referred to as an agent or LLM agent) may be “an expert” on a particular subject matter area and configured to complete a specific task or set of tasks related to that subject matter area. Further, the LLM may be a generative AI tool or a skill. For example, an LLM may utilize a trained skill that has access to a set of tools (e.g., API calls) used to accomplish the functions for which it is trained. LLMs can provide input to and receive output from other machine-learning models. For example, an LLM may send its output to a linear regression machine-learning model trained for a specialized prediction task and receive a prediction based on the input. In another example, an LLM may query a lightweight small language model that is specialized for particular tasks. Combining different architectures of LLMs, tools, and agents, among other architectures, can reduce computation and latency across the agentic system 120. The agentic system 120 may include an intake agent, baseline agent, planner agent, and reporter agent that are components capable of each applying one or more of LLMs to accomplish one or more tasks.

Along with the intake agent, baseline agent, planner agent, and reporter agent, the agentic system 120 may include additional or alternative agents. For example, the agentic system 120 may include a full agent that is configured to act as a health coach based on received interactions, a context agent that provides responses based on a set of context when specialized abilities of another agent is not required, and a research agent configured to provide responses to health inquiries based on medical research and scientific sources. Other example agents include a reports agent that is configured to generate progress reports and insights and a workout agent that is configured to facilitate exercise programming The router agent may be incorporated in one or more of the agents or may be a separate orchestrator for routing between agents.

The agentic system 120 receives indications to determine one or more wellness actions for a user. The agentic system 120 may receive the indication from a user device 110 in response to a user or user's health advisor (e.g., doctor, coach, etc.) requesting one or more wellness actions via a user interface 115. The agentic system 120 may also generate indications that cause itself to autonomously determine wellness actions for a user at periodic intervals, in response to detecting specific data within the user's user wellness data, etc.

Task determination module 121 determines which tasks are required to generate the wellness action based on the natural language query. The query to generate the wellness action may include context that indicates which tasks are required to generate the wellness action. In determining the wellness action, a task that is generated may require a sequence of supporting tasks to determine the wellness action. For example, the user may request that the agentic system 120 provides a series of wellness actions for the purpose of reducing their mile time when running. Task determination module 121 may determine that three primary tasks will be useful for reducing the user's mile time. The three tasks might include accessing a historical database containing previous mile times, accessing dietary records, and generating a workout plan.

Task determination module 121 breaks the request into a manageable number of tasks that guide the generation of wellness actions downstream. As such, task determination module 121 extracts signals from the natural language query to determine which tasks should be generated to generate the wellness action. Task determination module 121 may include an LLM (e.g., a task determination agent) that receives the query and outputs a set of tasks to determine the wellness action. Example signals include keywords related to candidate tasks and predetermined phrases that are associated with candidate tasks. In some embodiments, the LLM determines which tasks should be generated based on historical user data (e.g., stored in the user wellness data repository 140). Tasks generated for the wellness action may be generated based on at least, the user's preferences and keywords from the query.

Context module 122 receives the tasks generated by the task determination module 121 and retrieves a relevant set of context from the agentic system 120 or a user wellness data repository 140. Context module 122 retrieves portions of the user wellness data repository 140 to filter the context used for downstream decision-making and actions taken by LLMs. Subsets of context retrieved from the context module 122 are allocated (by the task-context determination module 124) and loaded into respective LLMs to accomplish tasks to which they are assigned. If the user wellness data repository 140 for a user was loaded as context for any one LLM, it would likely exceed the context window of the underlying model. An LLM contained in the context module 122 can access a database describing the user wellness data repository 140 stored in an organized structure (e.g., an indexed database). As such, the LLM can access the structure without loading the entire user wellness data repository 140 into its context window.

Context retrieved by the context module 122 includes portions of the user wellness data repository 140 associated with different facets of the user's wellness. For example, one portion may contain all of a user's exercise logs and another might include their dietary information and associated data (e.g., meal plan tracking).

The context module 122 can retrieve user information from user data stored at the agentic system 120, data that the agentic system 120 requests from a user device 110, the user wellness data repository 140, or any other suitable information. As such, the set of context may be determined by the LLM of the context module 122 before any LLM is associated with a task. Context module receives the tasks generated by the task determination module 121 and determines relevant portions of the user wellness data repository 140 for later use by downstream agents. For example, the LLM may receive a prompt comprising one or more tasks and instructions to determine portions of the user wellness data repository 140 relevant to completing the task. With the database representing the user wellness data repository 140 loaded as context in its context window, the LLM outputs a response containing one or more relevant portions of the user wellness data repository 140.

In some embodiments, context module 122 retrieves the set of context based on heuristics. Upon receiving a task, such as accessing a historical database containing previous mile times, context module 122 determines which portions of the database are relevant to the task. Based on the relationship between a mile time and exercise logs, context module 122 may determine that the exercise logs for the user are to be included in the set of context. As such, portions unrelated to the task (e.g., results from a hearing exam or historical records pertaining to insomnia) are not included in the set of context. The database describing the user wellness data repository 140 may be prelabeled, such that the context of each portion does not have to be included in the context window of the LLM of the context module 122. For example, the LLM may access a database that has previously been labeled by a machine-learning model trained to classify wellness data according to a set of labels.

Heuristic-based retrieval includes associating tasks (or keywords associated with tasks) with specific portions of the user wellness data repository 140. For example, tasks related to a user's diet might always require retrieving dietary logs from the user wellness data repository 140. In embodiments where tasks received from the task determination module 121 are classified according to a set of labels, each task may be associated with portions of the user wellness data repository 140. For example, rules for retrieval may be stored in a lookup table containing a set of task labels and associated relevant portions of the user wellness data repository 140.

Routing module 123 receives the set of tasks from the task determination module 121 and determines which LLM(s) should be assigned to each task. The agentic system 120 includes a plurality of specialized LLMs that are tailored to accomplishing a variety of tasks. Assigning a task to an agent or LLM without consideration for its specialization and training is computationally inefficient and may result in unwanted outcomes such as hallucinations. As such, routing module 123 includes context such as descriptions of the tasks and descriptions of the specialized LLMs to allocate appropriate tasks to LLMs of the agentic system 120.

Routing module 123 interfaces with an LLM (e.g., a routing agent) that receives the set of tasks and assigns LLMs to each task. The agentic system 120 uses the system of LLMs to complete one or more tasks for the agentic system 120. Routing module 123 receives a routing prompt comprising the set of tasks, descriptions of each of the LLM's capabilities, and instructions to allocate the set of tasks to one or more available LLMs to each task. Responsive to receiving the routing prompt, the LLM generates a list of suitable LLMs corresponding to each task.

Further, routing module 123 may generate a sequential plan to complete the set of tasks and an assignment of LLMs to complete each task. As such, LLMs pass on their outputs to other LLMs in a strategic way such that the LLMs work together to complete complex tasks. For each task, routing module 123 determines which LLM of the system of LLMs to send the query to. In some embodiments, the first LLM is a router agent that is configured to break the task into a set of subtasks that must all be completed to complete the task itself and determine how to route the query through one or more LLMs to complete the task. For each subtask, the router agent may assign the subtask to an LLM based on a description of what the LLM is capable of doing. The router agent creates a prompt that indicates to a first LLM assigned to a first task what the task is and which LLM to send its output to. The prompt may also include an order to apply LLMs to outputs of other LLMs to complete the task. For instance, the prompt may indicate for the first LLM to complete a first subtask, for the first LLM to send its output to a second LLM for a second subtask, and for the second LLM to send its output to a third LLM for a final subtask. The LLMs may be configured to access data needed to complete respective subtasks or may be instructed to provide such data for the next LLM in the route in the prompts that are output.

In some embodiments, routing module 123 determines whether completion of a task requires the output of a preceding task, based on the task description. By determining the dependencies of a task, routing module 123 generates a plan that considers which tasks can be completed in parallel and which tasks must be performed sequentially. For example, routing module 123 may assign a task to retrieve exercise records to a first search LLM and a task to retrieve dietary records to a second search LLM. Based on the descriptions of these respective tasks, routing module 123 determines that the assigned LLMs can perform their tasks in parallel. In another example, where user records are not yet populated in the user wellness data repository 140, routing module 123 would determine that retrieval task assigned to a search LLM depends on a wellness data collection task assigned to an intake LLM.

In some embodiments, each LLM includes its own subrouter agent that enables the LLMs to complete the task together. The subrouter agents may be part of the LLM or a sub-LLM of the LLM. For instance, the router agent may input a first prompt to a first LLM. The first prompt may indicate what the task is, and the first router agent at the first LLM may determine a first subtask for the first LLM to perform. The first LLM provides a first output based on the first subtask. The first LLM's subrouter agent determines a second LLM to complete a second subtask for the task and accesses data the second LLM needs to complete the second subtask. The first subrouter agent creates a prompt including the accessed data and the first output and sends the prompt to the second LLM. The second LLM performs the subtask and a second router agent at the second LLM receives a second output from the second LLM. The second router agent determines a third LLM to perform a third subtask and creates a prompt using the second output and accessed data for the third subtask. This process of routing subtasks to LLMs may continue until the task is complete, and any resulting data or an indication that the task was completed may be returned to the router agent.

Task-context determination module 124 receives the set of context from the context module 122 and determines which subsets of context should be allocated to each LLM to perform each task. Context module 122 includes an LLM that receives the set of tasks (from the task determination module 121), the set of LLMs associated with the tasks (from the routing module 123), and set of context (from the context module 122) as input and associates each LLM with a subset of relevant context data needed to perform the tasks to which it is assigned.

In some embodiments, the subset of context comprises content from the portion of the user wellness data repository 140. As such, task-context determination module 124 receives the portion of the user wellness data repository 140 identified by the context module 122 and extracts content from the portion. For example, based on context module 122 determining that the user's exercise logs are relevant to improving their mile time, task-context determination module 124 accesses the data of the exercise logs to load as context for an LLM associated with the task. As such, the LLM performing the task has access to source-of-truth or original, uncompressed data without loading the entire user wellness data repository 140 into its context window.

For the example of a task to access a historical record of mile times for the user, task-context determination module 124 allocates subsets of context that are needed for agents associated with the task. As such, the context LLM would allocate the portion of the user wellness data repository 140 containing exercise logs to the search LLM, but not a motivational speech LLM. Selectively allocating subsets of context to each LLM reduces the context window of all LLMs. In some embodiments, some subsets of context may have overlap, where the same context may be needed for multiple LLMs. However, the specialized nature of each LLM leads to an outcome where the entire context is not allocated to any one LLM. In some embodiments, the agentic system 120 persistently stores user context retrieved by an LLM to accomplish a task (e.g., a search LLM). Persistently storing user context facilitates quick retrieval of information that has already been processed by an LLM.

The context LLM may determine what set of context corresponds to each task. For example, the context LLM may take each task and prompt a large language model (e.g., a context agent) to segment a large set of contexts into subsets, each subset corresponding to a different task. In some embodiments, each agent assigned to the set of tasks receives an initial subset of context for performing the tasks. As tasks are completed, portions or all of the subset of context can be passed to following LLMs. LLMs can determine which portions of their associated subset of context is to be passed to following LLMs. In this process, relevant portions of a preceding LLM's context are loaded into the context window of a following LLM. As such, LLMs of the agentic system 120 may have a combination of a preceding LLM's context and the subset of context determined by the task-context determination module 124 loaded into its context window.

In some embodiments, each LLM accesses needed context data before processing its task. Task-context determination module 124 may provide context retrieved from the output of another LLM to an LLM associated with an incomplete task. For example, the first LLM processes a first task of the set of tasks using a first set of context data associated with the first task. The first LLM (or another part of the agentic system 120) sends a first results from the processing of the first task to a second LLM. The second LLM processes a second task of the plurality of tasks using the first results and a second set of context data associated with the second task. The second LLM sends a second result from the processing of the second task to a third LLM. The third LLM processes a third task of the plurality of tasks using the second results and a third set of context data associated with the third task and the second result. The third LLM (or another component of the agentic system 120) transmits, for display via a user interface 115 of a user device 110, a third result from processing of the third task. As such, the routing agent is not required to determine subsets of context after the first LLM has loaded its corresponding context and output a determination.

In some embodiments, task-context determination module 124 determines subsets of context to be provided to an LLM based on dependencies between tasks. Task-context determination module 124 may allocate subsets of context according to the task dependencies received from the routing module 123. As such, task-context determination module 124 may identify that a subset of context is only relevant to a downstream LLM as it relates to the output of a preceding LLM. In some embodiments, this dependency is determined based on the description of the task and the description of an LLM. For example, the subset of context to be provided to the reporter agent may be determined to be dependent on the output of the planner agent. In some embodiments, task-context determination module 124 determines which subsets of context are to be allocated within a temporal window based on, at least, the output of the routing module 123 describing which tasks can be completed in parallel and which tasks must be performed sequentially.

Visualization module 125 generates visual output for the user based on the one or more generated wellness actions. As wellness actions are generated and stored, visualization module 125 may display portions of the program at a user device 110, etc. To do so, visualization module 125 presents media content provided by the agentic system 120. Visualization module 125 may generate user interface elements that representative of or associated with the wellness actions. For example, visualization module may generate user interface elements for display that are associated with the user's identifier, the time period, the goal date, determinations of level of completion of the wellness action, and associated user wellness data.

### Example Agent Implementation Details

The router agent of the agentic system 120 may receive a request for a wellness program of wellness actions for a user to complete. The agentic system 120 determines that the user has not completed a motivational interview yet and sends a prompt to the intake agent to conduct a motivational interview with the user to gather wellness data. Once the intake agent has completed its subtask (e.g., conducting the interview), an intake subrouter agent of the intake agent sends the transcript of the motivational interview to the baseline agent. A baseline subrouter agent receives the transcript and accesses wellness data for the user that may be stored at the agentic system 120, such as wellness data the user entered when making an account. The baseline subrouter agent creates a prompt with the transcript and wellness data and inputs the prompt to the baseline agent. The baseline agent determines a baseline for the user, and the baseline subrouter agent sends the baseline to the planner agent. A planner subrouter agent receives the baseline and accesses one or more framework and one or more sets of principles that the planner subrouter agent includes in a prompt with the baseline. The planner agent receives the prompt and outputs a program of wellness actions for the user, which the planner subrouter sends back to the router agent, which may store the program, display one or more portions of the program at a user device 110, etc.

In some embodiments, a router agent, rather than subrouter agents, tracks the processing of the subtasks through the assigned LLMs and may store data describing the outputs. If an LLM is unable to process a prompt or does not output a prompt, the router agent may create a new prompt for the LLM based on the output from the last previous LLM in the route. In some embodiments, to handle instances when a respective LLMs is unable to properly process its subtask, the prompt may also include an indication to send the prompt input to the respective LLM and a description of its inability to process the prompt back to the router agent. In some embodiments, the router agent uses the prompt to determine the subtasks that have been completed and the subtasks that were not completed and create a new route of subtasks assigned to LLMs. In some embodiments, the router agent sends a notification of the LLM's inability to the user device 110 that provided the query.

The intake agent may be configured to capture user wellness data and determine inferred characteristics of an associated user. The intake agent may send communications to the user (via the user device 110) to conduct a motivational interview with the user. The intake agent may use one or more LLMs that determine communications that respond to the user's responses during the interview and focus the interview on retrieving information from the user that is specified by an external operator or that is missing from the user wellness data. For example, the intake agent may request information about what the user's goals are, why the user started interacting with the agentic system 120, what the user's limits are, etc. The intake agent stores a transcript of the interview in association with an identifier of the user and the user's user wellness data.

The intake agent may also use the transcript to determine inferred characteristics of the user. A user's inferred characteristics may describe attributes of the user's personality, habits, and limits, which can include limits of what the user is physically capable of and actions the user is or is not willing to take. For example, a user's inferred characteristics may describe that a user is motivated by encouragement from a coach, is a late riser and works out in the evenings, does not like to eat broccoli, cannot run for more than 10 minutes straight, does not want to use a treadmill, etc. The intake agent may generate a prompt that includes the transcript of the user's motivation interview, wellness data extracted from the transcript, and/or wellness data for the user already stored at the agentic system 120 and inputs the prompt to one or more LLMs or other machine learning models trained to output inferred characteristics of a user. In some embodiments, the prompt also includes other data related to the user, such as communication records between a user and other users or with one or more coaches, a user's calendar, a user's location data and weather associated with the location(s), etc. The intake agent stores inferred characteristics in association with the user's wellness data.

The baseline agent may receive inferred characteristics of a user from the intake agent. The baseline agent is configured to determine a baseline for a user based on the user's wellness data and inferred characteristics. A user's baseline describes the user's physical abilities and health metrics-for example, the physical abilities may include how much weight a user can bench, how fast the user can run, how often the user works out, how many steps they typically take throughout the day, how much they sleep, etc. and the user's health metrics may include a user's average resting heart rate, average glucose levels over time, how much medicine or supplements the user takes, etc. The baseline agent creates a prompt including the inferred characteristics and user's wellness data and inputs the prompt to one or more LLMs configured to output a baseline for the user. In some embodiments, the baseline agent also includes the transcript of the motivation interview in the prompt. The baseline agent stores the user's baseline in association with the user's wellness data.

The planner agent may receive the baseline from the baseline agent. In some embodiments, the planner agent also receives and the inferred characteristics from the intake or planner agent. The planner agent is configured to determine one or more wellness actions for the user to take. The planner agent may access one or more frameworks that outline a generic program of wellness actions for users to take based on their goals, medical guidelines, or scientific results. The planner agent may also access one or more sets of wellness principles that define healthy practices for users to implement into their lives to improve their health, which may also be based on medical guidelines or scientific results. The planner agent may select the framework(s) and set(s) of wellness principles to correspond to the baseline of the user. For example, if the user's baseline indicates that the user has poor work-life balance habits, the planner agent may access a set of principles that indicate to manage time effectively, learn to say no, set clear boundaries, etc.

The planner agent creates a prompt including the baseline, the one or more frameworks, and the one or more sets of frameworks. In some embodiments, the planner agent automatically includes particular frameworks or sets of principles that all users may benefit from following. For example, the planner agent may always include principles about sleep health. The planner agent inputs the prompt to one or more LLMs that are configured to determine wellness actions for a user based on a prompt. In some embodiments, the planner agent outputs the wellness actions in a scheduled program that indicates the time period and goal date of each wellness action.

The planner agent may receive reflection data from user devices 110 upon reaching the end of a time period of a scheduled program of wellness actions (e.g., at the end of the week). The reflection data may include user wellness data describing what wellness actions the user completed, in whole or in part, what other activity the user has done during the time period, explanations from the user about why she did or did not complete particular wellness actions, a set of new goals input by the user, etc. The planner agent may request a new baseline from the intake agent and create a new prompt with the new baseline, the reflection data, and one or more frameworks or sets of principles. The planner agent may determine a new scheduled wellness actions for the user and continue to create a new scheduled program at the end of a time period of a previous scheduled program.

The reporter agent receives wellness actions from the planner agent. In some embodiments, the reporter agent may track the user's activity in relation to a time period of a scheduled program of wellness actions. The reporter agent may input the wellness actions (or scheduled program) to one or more LLMs configured to output one or more notifications and requests for the reporter agent to send to the user during one or more sub-time periods of the time periods associated with the wellness actions. Over each sub-time period (e.g., a day), the reporter agent may send the notifications or requests to a user device 110 of the user. The notifications may include reminders of wellness actions in the scheduled program and may be sent in accordance with the timing outlined in the scheduled program. The requests may ask the user to input information describing what activities she has done for the sub-time period and how the user has felt during the time-period. The reporter agent may use the user's responses to the requests in a new prompt of wellness actions for the next sub-time period such that the reporter agent may receive a new set of notifications and requests to send to the user that incorporates the user's responses. The reporter agent may input new prompts at the end of each sub-time period and may also store the user's responses as reflection data that the planner agent may use.

In some embodiments, more or less LLM agents than those described above are used to process the set of tasks. Further, in some embodiments, each respective LLM sends its results from the processing of its respective task to a respective next LLM agent, along with results from any respective previous LLMs, thus resulting in each LLM receiving the results of each LLM that performed processing before. In some embodiments, the results are stored at the agentic system such that each LLM may access the results when retrieving context data for its task. In some embodiments, the first LLM determines the set of tasks and a route of the tasks through the LLM system and causes each LLM to send the set of tasks and route along with the results of its processing to its respective next LLM.

### Example Method

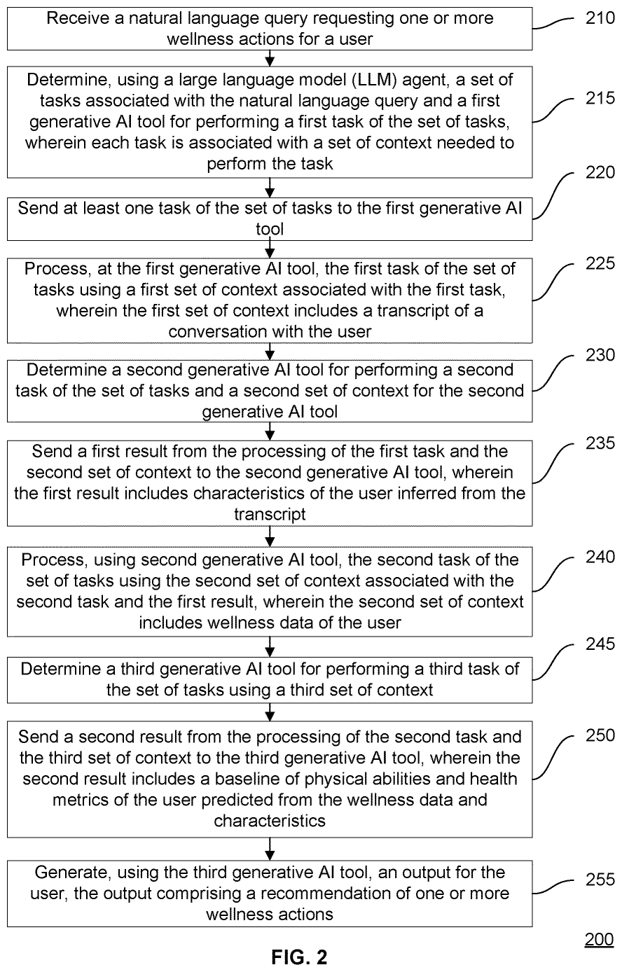

FIG. 2 is a flowchart showing an exemplary process 200 for deploying a personalized wellness agent, in accordance with one or more embodiments. As depicted in FIG. 2, process 200 may be run based on one or more processors executing instructions stored in a non-transitory computer-readable medium. As depicted in FIG. 2, process 200 may begin with agentic system 120 receiving 210 a natural language query requesting one or more wellness actions for a user. Agentic system 120 determines 215 (e.g., using task determination module 121), using an LLM agent, a set of tasks associated with the natural language query and a first generative AI tool for performing a first task of the set of tasks, wherein each task is associated with a set of context needed to perform the task (e.g., using context module 122). Agentic system 120 sends 220 (e.g., using routing module 123) at least one task of the set of tasks to the first generative AI tool and processes 225 (e.g., using task-context determination module 124), at the first generative AI tool, the first task of the set of tasks using a first set of context associated with the first task, wherein the first set of context includes a transcript of a conversation with the user. Agentic system 120 determines 230 (e.g., using routing module 123) a second generative AI tool for performing a second task of the set of tasks and a second set of context for the second generative AI tool (e.g., using task-context determination module 124). Agentic system 120 sends 235 a first result from the processing of the first task and the second set of context (e.g., using task-context determination module 124) to the second generative AI tool, wherein the first result includes characteristics of the user inferred from the transcript. Agentic system 120 processes 240, using second generative AI tool, the second task of the set of tasks using the second set of context associated with the second task and the first result, wherein the second set of context includes wellness data of the user. Agentic system 120 determines 245 (e.g., using routing module 123) a third generative AI tool for performing a third task of the set of tasks using a third set of context. Agentic system 120 sends 250 a second result from the processing of the second task and the third set of context (e.g., using task-context determination module 124) to the third generative AI tool, wherein the second result includes a baseline of physical abilities and health metrics of the user predicted from the wellness data and characteristics. Agentic system 120 generates 255 (e.g., using visualization module 125), using the third generative AI tool, an output for the user, the output comprising a recommendation of one or more wellness actions.

In various embodiments, a wide variety of machine learning techniques may be used for the machine-learning model described above. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN), long short-term memory networks (LSTM), transformers, linear recurrent neural networks such as Mamba may also be used.

In various embodiments, the training techniques for a machine learning model may be supervised, semi-supervised, or unsupervised. In supervised learning, the machine learning models may be trained with a set of training samples that are labeled.

By way of example, the training set may include multiple past image data labeled with known outcomes. Each training sample in the training set may correspond to a past and the corresponding outcome may serve as the label for the sample. A training sample may be represented as a feature vector that includes multiple dimensions. Each dimension may include data of a feature, which may be a quantized value of an attribute that describes the past record.

In some embodiments, an unsupervised learning technique may be used. The training samples used for an unsupervised model may also be represented by features vectors, but may not be labeled. Various unsupervised learning techniques such as clustering may be used in determining similarities among the feature vectors, thereby categorizing the training samples into different clusters. In some cases, the training may be semi-supervised with a training set having a mix of labeled samples and unlabeled samples.

A machine learning model may be associated with an objective function, which generates a metric value that describes the objective goal of the training process. The training process may intend to reduce the error rate of the model in generating predictions. In such a case, the objective function may monitor the error rate of the machine learning model. In a model that generates predictions, the objective function of the machine learning algorithm may be the training error rate when the predictions are compared to the actual labels. Such an objective function may be called a loss function. Other forms of objective functions may also be used, particularly for unsupervised learning models whose error rates are not easily determined due to the lack of labels. In various embodiments, the error rate may be measured as cross-entropy loss, L1 loss (e.g., the sum of absolute differences between the predicted values and the actual value), L2 loss (e.g., the sum of squared distances).

### Computer Architecture

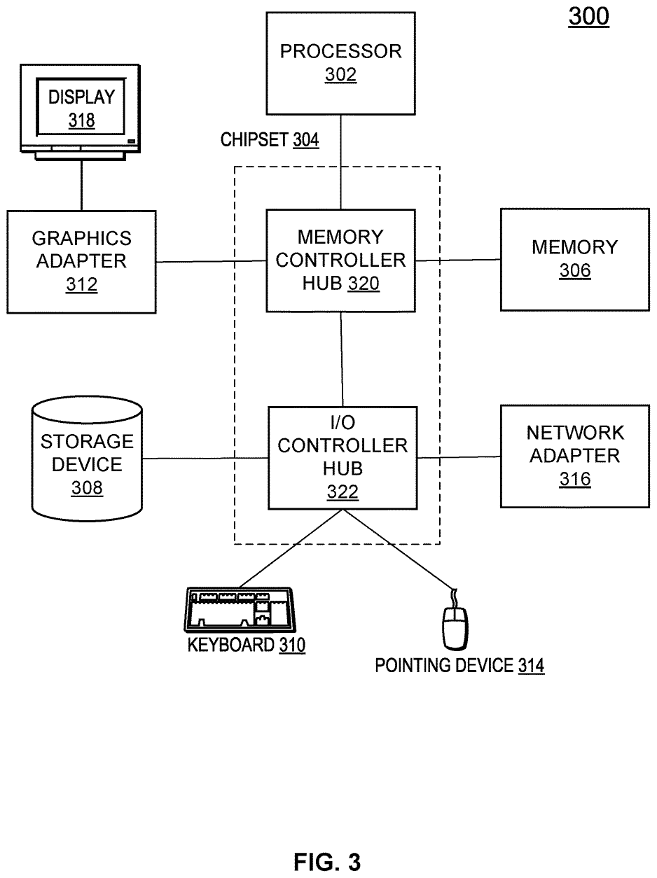

FIG. 3 is a high-level block diagram of a computer 300 for implementing different entities illustrated in FIGS. 1A and 1B, in accordance with one or more illustrative embodiments.
The computer 300 includes at least one processor 302 coupled to a chipset 304. Also coupled to the chipset 304 are a memory 306, a storage device 308, a keyboard 310, a graphics adapter 312, a pointing device 314, and a network adapter 316. A display 318 is coupled to the graphics adapter 312. In one embodiment, the functionality of the chipset 304 is provided by a memory controller hub 320 and an I/O controller hub 322. In another embodiment, the memory 306 is coupled directly to the processor 302 instead of the chipset 304.

The storage device 308 is any non-transitory computer-readable storage medium, such as a hard drive, compact disk read-only memory (CD-ROM), DVD, or a solid-state memory device. The memory 306 holds instructions and data used by the processor 302. The pointing device 314 may be a mouse, track ball, or other type of pointing device, and is used in combination with the keyboard 310 to input data into the computer system 300. The graphics adapter 312 displays images and other information on the display 318. The network adapter 316 couples the computer system 300 to the network 130.

As is known in the art, a computer 300 can have different and/or other components than those shown in FIG. 3. In addition, the computer 300 can lack certain illustrated components. For example, the computer acting as the agentic system 120 can be formed of multiple blade servers linked together into one or more distributed systems and lack components such as keyboards and displays. Moreover, the storage device 308 can be local and/or remote from the computer 300 (such as embodied within a storage area network (SAN)).

As is known in the art, the computer 300 is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device 308, loaded into the memory 306, and executed by the processor 302.

### Alternative Embodiments

The features and advantages described in the specification are not all inclusive and in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the disclosed subject matter.

It is to be understood that the figures and descriptions have been simplified to illustrate elements that are relevant for a clear understanding of the present invention, while eliminating, for the purpose of clarity, many other elements found in a typical online system. Those of ordinary skill in the art may recognize that other elements and/or steps are desirable and/or required in implementing the embodiments. However, because such elements and steps are well known in the art, and because they do not facilitate a better understanding of the embodiments, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the various embodiments. This description should be read to include one or at least one and the singular also includes the plural unless it is obvious that it is meant otherwise.

Upon reading this disclosure, those of skill in the art will appreciate still additional alternative designs for a unified communication interface providing various communication services. Thus, while particular embodiments and applications of the present disclosure have been illustrated and described, it is to be understood that the embodiments are not limited to the precise construction and components disclosed herein and that various modifications, changes and variations which will be apparent to those skilled in the art may be made in the arrangement, operation and details of the method and apparatus of the present disclosure disclosed herein without departing from the spirit and scope of the disclosure as defined in the appended claims.

What is claimed:

1. A method for deploying a personalized wellness agent, the method comprising:
receiving a natural language query requesting one or more wellness actions for a user;
determining, using a large language model (LLM) agent, a set of tasks associated with the natural language query and a first generative AI tool for performing a first task of the set of tasks, wherein each task is associated with a set of context needed to perform the task;
sending at least one task of the set of tasks to the first generative AI tool;
processing, at the first generative AI tool, the first task of the set of tasks using a first set of context associated with the first task, wherein the first set of context includes a transcript of a conversation with the user;
determining a second generative AI tool for performing a second task of the set of tasks and a second set of context for the second generative AI tool;
sending a first result from the processing of the first task and the second set of context to the second generative AI tool, wherein the first result includes characteristics of the user inferred from the transcript;
processing, using second generative AI tool, the second task of the set of tasks using the second set of context associated with the second task and the first result, wherein the second set of context includes wellness data of the user;
determining a third generative AI tool for performing a third task of the set of tasks using a third set of context;
sending a second result from the processing of the second task and the third set of context to the third generative AI tool, wherein the second result includes a baseline of physical abilities and health metrics of the user predicted from the wellness data and characteristics; and
generating, using the third generative AI tool, an output for the user, the output comprising a recommendation of one or more wellness actions.

2. The method of claim 1, wherein generating, using the third generative AI tool, the output for the user comprises:
processing, using the third generative AI tool, the third task of the set of tasks using the third set of context associated with the third task and the second result, wherein the third set of context includes a program framework; and
transmitting, for display via a user interface, a third result from processing of the third task, wherein the third result comprises the one or more wellness actions structured according to the program framework.

3. The method of claim 1, wherein the set of tasks associated with the natural language query requesting one or more wellness actions for a user are determined by:
receiving, from the LLM agent, the set of tasks associated with the natural language query;
sending a routing prompt to the first generative AI tool, the routing prompt comprising the set of tasks and instructions to allocate the set of tasks to one or more other generative AI tools; and
receiving, from the first generative AI tool, an assignment showing which of the one or more other generative AI tools is to perform each respective task.

4. The method of claim 3, wherein the first generative AI tool provides, to the third generative AI tool, context to complete the third task by:
receiving an indication that the second generative AI tool has completed the second task;
responsive to receiving the indication that the second generative AI tool has completed the second task, determining updated context for the third set of context, based on the second result, to be loaded for the third generative AI tool to complete the third task; and
loading the third set of context having the updated context for the third generative AI tool.

5. The method of claim 1, wherein the set of context needed to perform each task of the set of tasks is determined by:
receiving an indication that at least one task of the set of tasks has been completed by a generative AI tool associated with the task;
responsive to receiving an indication that task has been completed, retrieving the set of context from the output of the generative AI tool associated with the completed task; and
providing, to one or more generative AI tools associated with an incomplete task, the set of context needed to perform the incomplete task.

6. The method of claim 1, wherein the LLM agent is configured to persistently store user context associated with a user and provide the user context to one or more generative AI tools by:
accessing context from a database containing user wellness data;
receiving an indication that a generative AI tool is beginning a given task;
responsive to receiving an indication that the generative AI tool is beginning the given task, determining a portion of the user context relevant to the given task;
generating a response associating the portion of the user context needed for the generative AI tool to perform the given task;
parsing the response for the user context; and
sending the user context to the one or more generative AI tools.

7. The method of claim 1, wherein a fourth result from a fourth generative AI tool is used by the third generative AI tool to generate the output, and wherein the LLM agent determines that the fourth generative AI tool can process a fourth task without requiring the second result by determining that the fourth task does not require context that depends on output of the second result, wherein the fourth result is generated in parallel with generation of the second result.

8. A non-transitory computer-readable medium comprising memory with instructions encoded thereon that, when executed by one or more processors, cause the one or more processors to perform operations, the instructions comprising instructions to:
receive a natural language query requesting one or more wellness actions for a user;
determine, using a large language model (LLM) agent, a set of tasks associated with the natural language query and a first generative AI tool for performing a first task of the set of tasks, wherein each task is associated with a set of context needed to perform the task;
send at least one task of the set of tasks to the first generative AI tool;
process, at the first generative AI tool, the first task of the set of tasks using a first set of context associated with the first task, wherein the first set of context includes a transcript of a conversation with the user;
determine a second generative AI tool for performing a second task of the set of tasks and a second set of context for the second generative AI tool;
send a first result from the processing of the first task and the second set of context to the second generative AI tool, wherein the first result includes characteristics of the user inferred from the transcript;
process, using second generative AI tool, the second task of the set of tasks using the second set of context associated with the second task and the first result, wherein the second set of context includes wellness data of the user;
determine a third generative AI tool for performing a third task of the set of tasks using a third set of context;
send a second result from the processing of the second task and the third set of context to the third generative AI tool, wherein the second result includes a baseline of physical abilities and health metrics of the user predicted from the wellness data and characteristics; and
generate, using the third generative AI tool, an output for the user, the output comprising a recommendation of one or more wellness actions.

9. The non-transitory computer-readable medium of claim 8, wherein the instructions to generate, using the third generative AI tool, the output for the user further comprises instructions to:
process, using the third generative AI tool, the third task of the set of tasks using the third set of context associated with the third task and the second result, wherein the third set of context includes a program framework; and
transmit, for display via a user interface, a third result from processing of the third task, wherein the third result comprises the one or more wellness actions structured according to the program framework.

10. The non-transitory computer-readable medium of claim 8, wherein the set of tasks associated with the natural language query requesting one or more wellness actions for a user are determined by:
receiving, from the LLM agent, the set of tasks associated with the natural language query;
sending a routing prompt to the first generative AI tool, the routing prompt comprising the set of tasks and instructions to allocate the set of tasks to one or more other generative AI tools; and
receiving, from the first generative AI tool, an assignment showing which of the one or more other generative AI tools is to perform each respective task.

11. The non-transitory computer-readable medium of claim 10, wherein the first generative AI tool provides, to the third generative AI tool, context to complete the third task by:
receiving an indication that the second generative AI tool has completed the second task;
responsive to receiving the indication that the second generative AI tool has completed the second task, determining updated context for the third set of context, based on the second result, to be loaded for the third generative AI tool to complete the third task; and
loading the third set of context having the updated context for the third generative AI tool.

12. The non-transitory computer-readable medium of claim 8, wherein the set of context needed to perform each task of the set of tasks is determined by:
receiving an indication that at least one task of the set of tasks has been completed by a generative AI tool associated with the task;
responsive to receiving an indication that task has been completed, retrieving the set of context from the output of the generative AI tool associated with the completed task; and
providing, to one or more generative AI tools associated with an incomplete task, the set of context needed to perform the incomplete task.

13. The non-transitory computer-readable medium of claim 8, wherein the LLM agent is configured to persistently store user context associated with a user and provide the user context to one or more generative AI tools by:
accessing context from a database containing user wellness data;
receiving an indication that a generative AI tool is beginning a given task;
responsive to receiving an indication that the generative AI tool is beginning the given task, determining a portion of the user context relevant to the given task;
generating a response associating the portion of the user context needed for the generative AI tool to perform the given task;
parsing the response for the user context; and
sending the user context to the one or more generative AI tools.

14. The non-transitory computer-readable medium of claim 8, wherein a fourth result from a fourth generative AI tool is used by the third generative AI tool to generate the output, wherein the LLM agent determines that the fourth generative AI tool can process a fourth task without requiring the second result by determining that the fourth task does not require context that depends on output of the second result, and wherein the fourth result is generated in parallel with generation of the second result.

15. A system comprising:
memory with instructions encoded thereon; and
one or more processors that, when executing the instructions, are caused to perform operations comprising:
receiving a natural language query requesting one or more wellness actions for a user;
determining, using a large language model (LLM) agent, a set of tasks associated with the natural language query and a first generative AI tool for performing a first task of the set of tasks, wherein each task is associated with a set of context needed to perform the task;
sending at least one task of the set of tasks to the first generative AI tool;
processing, at the first generative AI tool, the first task of the set of tasks using a first set of context associated with the first task, wherein the first set of context includes a transcript of a conversation with the user;
determining a second generative AI tool for performing a second task of the set of tasks and a second set of context for the second generative AI tool;
sending a first result from the processing of the first task and the second set of context to the second generative AI tool, wherein the first result includes characteristics of the user inferred from the transcript;
processing, using second generative AI tool, the second task of the set of tasks using the second set of context associated with the second task and the first result, wherein the second set of context includes wellness data of the user;
determining a third generative AI tool for performing a third task of the set of tasks using a third set of context;
sending a second result from the processing of the second task and the third set of context to the third generative AI tool, wherein the second result includes a baseline of physical abilities and health metrics of the user predicted from the wellness data and characteristics; and
generating, using the third generative AI tool, an output for the user, the output comprising a recommendation of one or more wellness actions.

16. The system of claim 15, wherein generating, using the third generative AI tool, the output for the user comprises:
processing, using the third generative AI tool, the third task of the set of tasks using the third set of context associated with the third task and the second result, wherein the third set of context includes a program framework; and
transmitting, for display via a user interface, a third result from processing of the third task, wherein the third result comprises the one or more wellness actions structured according to the program framework.

17. The system of claim 15, wherein the set of tasks associated with the natural language query requesting one or more wellness actions for a user are determined by:
receiving, from the LLM agent, the set of tasks associated with the natural language query;
sending a routing prompt to the first generative AI tool, the routing prompt comprising the set of tasks and instructions to allocate the set of tasks to one or more other generative AI tools; and
receiving, from the first generative AI tool, an assignment showing which of the one or more other generative AI tools is to perform each respective task.

18. The system of claim 17, wherein the first generative AI tool provides, to the third generative AI tool, context to complete the third task by:
receiving an indication that the second generative AI tool has completed the second task;
responsive to receiving the indication that the second generative AI tool has completed the second task, determining updated context for the third set of context, based on the second result, to be loaded for the third generative AI tool to complete the third task; and
loading the third set of context having the updated context for the third generative AI tool.

19. The system of claim 15, wherein the set of context needed to perform each task of the set of tasks is determined by:
receiving an indication that at least one task of the set of tasks has been completed by a generative AI tool associated with the task;
responsive to receiving an indication that task has been completed, retrieving the set of context from the output of the generative AI tool associated with the completed task; and
providing, to one or more generative AI tools associated with an incomplete task, the set of context needed to perform the incomplete task.

20. The system of claim 15, wherein the LLM agent is configured to persistently store user context associated with a user and provide the user context to one or more generative AI tools by:
accessing context from a database containing user wellness data;
receiving an indication that a generative AI tool is beginning a given task;
responsive to receiving an indication that the generative AI tool is beginning the given task, determining a portion of the user context relevant to the given task;
generating a response associating the portion of the user context needed for the generative AI tool to perform the given task;
parsing the response for the user context; and
sending the user context to the one or more generative AI tools.
